(19) United States
(12) Patent Application Publication
(10) Pub. No.: US 2012/0107395 A1
(43) Pub. Date: May 3, 2012

(54) Probiotic Soft Gel Compositions

(75) Inventors: Xueju Xie, Richmond (CA); Jason Jiang-Chung Ko, Richmond (CA)

(73) Assignee: VIVA PHARMACEUTICAL INC., Richmond (CA)

(21) Appl. No.: 12/962,556

(22) Filed: Dec. 7, 2010

Related U.S. Application Data
(60) Provisional application No. 61/409,046, filed on Nov. 1, 2010.

(51) Int. Cl. A61K 9/48; A61P 1/00; A61K 35/74

(52) U.S. Cl. 424/452; 424/93.42; 424/93.4

(57) ABSTRACT

Soft gel capsules are formed from an outer gelatin-containing shell and a filling. The filling includes a probiotic and a dessicant.

### FIELD

This disclosure relates to soft gel capsules. In particular, this disclosure relates to soft gel capsules comprising a probiotic. This disclosure further relates to methods, processes, kits, and the like.

### BACKGROUND

Oral dosage forms such as tablets, pills, and capsules are used for a variety of purposes. For example, administration of pharmacologically active agents, nutraceuticals, vitamins and other supplements, oral hygiene products, foodstuffs, and other ingredients of interest. Such dosage forms are generally convenient, stable in storage and transport, and familiar to the user.

Soft gel capsules are one preferred oral dosage form. These capsules are often viewed by consumers as an efficient method of delivery for the ingredient of interest and can be more palatable especially for those consumers who have difficulty swallowing hard tablet or pills. Typically, soft gel capsules are made from aqueous solutions of gelling agents like gelatin or plant polysaccharides (e.g. carrageenans). Other ingredients may be added such as, for example, plasticizers, flavours, colouring agents, preservatives, disintegrants, lubricants, and the like.

Probiotics are live microorganisms which when administered in adequate amounts may confer a health or other type of benefit to the host. Probiotics are well described in the art. See, for example, the Health Canada Monograph entitled “Probiotics” dated Feb. 11, 2009. The market for probiotics is rapidly growing and several successful mass-marketed products exist such as Yakult™ and Activa™. Probiotics are living microorganisms and consequently present a special challenge from a manufacturing and formulation standpoint. In particular, probiotics can be very sensitive to moisture content, mechanical stress, other ingredients in the formulation, etc. To date manufacturing soft gel capsules containing probiotics has proved problematic. It is believed that the relatively high moisture content during the encapsulation process has a deleterious effect on the number of colony forming units (CFU) remaining after processing. Furthermore, the mechanical stresses of soft gel manufacturing have a detrimental effect on certain probiotics.

It would be advantageous to produce soft gel capsules comprising a probiotic composition in a manner which maintained as many CFUs as possible.

### SUMMARY

The present disclosure provides a soft gel capsule comprising a probiotic and a desiccant.

The present disclosure provides a method of manufacturing a soft gel capsule comprising a probiotic. For example, the present method may maintain about 50% or greater of the CFUs.

The present disclosure further provides compositions, uses, and processes relating to probiotic soft gel capsules.

As used herein, the term “soft gel capsule” means an oral dosage form having an outer shell composition comprising a gelling agent and a filling composition comprising a probiotic.

As used herein, the term “probiotic” means live microorganisms which when administered in adequate amounts may confer a health benefit to the subject.

As used herein, the term “subject” is not limited to a specific species or sample type. For example, the term “subject” may refer to a patient, and frequently a human patient. However, this term is not limited to humans and thus encompasses a variety of mammalian species.

As used herein, “a” or “an” means “at least one” or “one or more”.

This summary does not necessarily describe all features of the invention. Other aspects, features and advantages of the invention will be apparent to those of ordinary skill in the art upon review of the following description of specific embodiments of the invention.

### DETAILED DESCRIPTION

The present disclosure provides soft gel capsules. Such capsules have been described in the art. See for example, U.S. Pat. No. 7,662,406; US2009/0208608; WO1993/004674. The term ‘soft gel’ generally refers to a one-piece, hermetically sealed soft gelatin outer shell containing a filling composition that is often oil, liquid, a suspension, or a semi-solid. Viscosity builder, or gelling agents other than gelatin may also be used.

The present soft gel capsule may comprise an outer shell and a filling. The outer shell composition may comprise a suitable amount of at least one gelatin. For example, the outer shell may comprise from about 10% to about 90%, from about 15% to about 80%, from about 20% to about 70%, from about 25% to about 60%, about 30% to about 50%, by weight, of gelatin. Particularly suitable gelatins include those derived from pigskin, bovine bone, or fish gelatin.

According to one aspect, the gelatin is selected from the group consisting of an animal-derived gelatin, a chemically modified gelatin, a physically modified gelatin, and combinations thereof.

The outer shell may comprise a variety of other ingredients. For example, a plasticizer, an anti-tack agents, a softening agent, a starch, a flavour, a colourant, a preservative, and combinations thereof.

The outer shell may optionally comprise at least one starch. For example, the shell may comprise starch in an amount selected from the range of 0.1% to 35% of the total weight of the shell composition. Any suitable starch may be used. The starch may be selected from native starches, modified starches, polysaccharides, and combinations thereof. Suitable native starches are exemplified by potato starches, corn starches, wheat starches, oat starch, barley starch, rice starches, sorghum starches, legume starch, pea bean starch, and tapioca starches. Modified starches are native starches that have been modified partially degraded by physical treatments or alternatively, by chemical treatments, and are commonly referred to as physically modified starches and chemically modified starches. Suitable physical treatments are exemplified by pre-gelatinization and by heat-moisture treatments. Suitable physically modified starches are exemplified by physically modified potato starches, physically modified corn starches, physically modified wheat starches, physically modified oat starch, physically modified barley starch, physically modified rice starches, physically modified sorghum starches, and physically modified tapioca starches. Suitable chemical treatments are exemplified by alkali washes, washes with inorganic acids, enzymatic hydrolysis, bleaching, oxidation, esterification, etherification, cross-linking, ionization, and combinations of these modifications such as acetylation and oxidation. Suitable chemically modified starches are exemplified by esterified starch, starch phosphate, etherified starches, cross-linked starches, cationized starches, enzymatically digested starches, oxidized starches, and combinations thereof.

The outer shell may optionally comprise an anti-tacking and/or softening agent. The agent(s) may be selected from lecithin, polysorbate such as Tween 60 or 80, biologically derived waxes, chemically derived waxes, fats, oils or combinations thereof. Some exemplary embodiments of the present invention relate to an anti-tacking and softening agent include beeswax, lecithin, palm oil and coconut oil.

The outer shell may optionally comprise an oil selected from palm oil, coconut oil, vegetable oil, middle chain triglycerins, and combinations thereof.

Some exemplary embodiments of the present invention relate to a chewable soft gel capsule exhibiting extended storage stability, a soft chewing texture and low stickiness. Further, the chewable soft gel capsule exhibits excellent manufacturing properties and can be produced using conventional encapsulation machinery known in the art.

Some exemplary embodiments of the present invention related to a soft gel capsule encasing an orally ingestible article, the capsule having an outer shell composition comprising at least (a) at least one gelatin, preferably between about 20 to about 60% weight, more preferably between about 20 to about 50% weight, even more preferably between about 30 to about 47% weight; (b) at least one plasticizer in an amount sufficient to render said outer shell flexible, preferably between about 25 to about 45% weight; (c) an anti-tacking and softening agent sufficient to render the outer shell non-sticky and soft, preferably about 0.2 to about 15% weight, more preferably between about 0.4 to about 10% weight, even more preferably about 0.6 to about 5% weight; and (d) purified water, preferably between about 3 to about 25% weight, more preferably between about 3 to about 20% weight. The capsule outer shell further includes (e) at least one starch, preferably between about 0.1 to about 35% weight, more preferably between about 2 to about 25% weight, even more preferably between about 10 to about 20% weight. The capsule outer shell may optionally further include (f) at least one of flavorants, colorants, and preservatives.

Some exemplary embodiments of the present invention relate to at least one plasticizer selected from the group consisting of glycerin, mannitol, polyethylene glycol, sorbitol, sorbitol special, propylene glycol, maltitol, sucrose, corn syrup, fructose, cellulose, disodium sulfosucciante, triethyl citrate, tributyl citrate, 1-2-propylenglycol, natural gum, isomerized sugar, xylitol, polyglycerol, glucose syrups, glucose, sugar alcohol, and combinations thereof.

Some exemplary embodiments of the present invention relate to a softgel capsule where the anti-tacking and softening agent is preferably between about 0.2 to about 15% weight, more preferably between about 0.4 to about 10% weight, and even more preferably between about 0.6 to about 5% weight.

The present soft gel capsules comprise a filling comprising a probiotic composition. Any suitable probiotic or mixture of probiotics may be used herein. Probiotic compositions are known in the art. See for example US2010/0221226; US2010/0266727; US2010/0266560; U.S. Pat. No. 6,060,050. Various probiotics are known such as, for example, Bifidobacterium adolescentis; fidobacterium animalis; Bifidobacterium bifidum; Bifidobacterium breve; Bifidobacterium infantis; Bifidobacterium lactis; Bifidobacterium longum; Lactobacillus acidophilus; Lactobacillus amylovorus; Lactobacillus casei; Lactobacillus fermentum; Lactobacillus gasseri; Lactobacillus johnsonii; Lactobacillus paracasei; Lactobacillus plantarum; Lactobacillus reuteri; Lactobacillus rhamnosus; Lactobacillus salivarius; Streptococcus thermophilus; Streptococcus Salivarius; Streptococcus oralis; Streptococcus uberis; Streptococcus rattus; Escherichia coli; Bacillus coagulans; Bacillus lansii; Saccharomyces cerevisiae; Saccharomyces boulardii; and combinations thereof. Preferred probiotics for use herein include Bifidobacterium longum; Streptococcus salivarius; and combinations thereof.

The activity of probiotics may be assessed in terms of CFUs. Techniques for assessing CFUs are well known. After manufacture, the present soft gel preparations preferably maintain at least about 50%, about 55%, about 60%, about 65%, about 70% about 75%, about 80%, of the CFUs of the probiotic.

The present filling comprises a desiccant. A desiccant may be defined as a hygroscopic substance that induces or sustains a state of dryness in its local vicinity. Any suitable desiccant may be used herein. For example, calcium sulfate; sodium sulfate; magnesium sulfate; dextrins; maltodextrin, polysaccharides; oligosaccharides; lecithin; silicas; bentonite clays; starches; and combinations thereof. The desiccant is preferably present in an amount of about 0.1% to about 75% by weight of the total filling composition.

Preferred are starches. The filling composition preferably comprises at least one starch in an amount selected from the range of about 0.1% to about 75% by weight of the total filling composition. Any suitable starch may be used. The starch may be selected from native starches, modified starches, polysaccharides, and combinations thereof. Suitable native starches are exemplified by potato starches, corn starches, wheat starches, oat starch, barley starch, rice starches, sorghum starches, legume starch, pea bean starch, and tapioca starches. Modified starches are native starches that have been partially degraded by physical treatments or alternatively, by chemical treatments, and are commonly referred to as physically modified starches and chemically modified starches. Suitable physical treatments are exemplified by pre-gelatinization and by heat-moisture treatments. Suitable physically modified starches are exemplified by physically modified potato starches, physically modified corn starches, physically modified wheat starches, physically modified oat starch, physically modified barley starch, physically modified rice starches, physically modified sorghum starches, and physically modified tapioca starches. Suitable chemical treatments are exemplified by alkali washes, washes with inorganic acids, enzymatic hydrolysis, bleaching, oxidation, esterification, etherification, cross-linking, ionization, and combinations of these modifications such as acetylation and oxidation. Suitable chemically modified starches are exemplified by esterified starch, starch phosphate, etherified starches, cross-linked starches, cationized starches, enzymatically digested starches, oxidized starches, and combinations thereof. The present starch may be selected from, for example, from potato starches, corn starches, wheat starches, oat starch, barley starch, rice starches, sorghum starches, legume starch, pea bean starch, tapioca starches, and combinations thereof. Preferred are potato starches, corn starches, tapioca starches, and combinations thereof.

The filling composition may optionally comprise an emulsifier such as, for example, lecithin, polysorbates, or combinations thereof.

The filling composition may optionally comprise an oil from any edible oils such as, for example, fish oil, krill oil, seal oil, borage oil, flax seed oil, evening primrose oil, soybean oil, hemp seed oil, pumpkin seed oil, grape seed oil, germ oil, safflower oil, squalene oil, squalane, sesame seed oil, arachidonic acid, conjugated linoleic acid, rosemary oil, lemon oil, peppermint oil, terrapin oil, pomegranate seed oil, sea buckthorn fruit oil, sunflower oil, chia oil, goji berry oil, sea buckthorn oil, jasmine oil, sweet almond oil, ginger oil, parsley seed oil, orange oil, halibut liver oil, wheat germ oil, aloe vera oil, garlic oil, avocado oil, castor oil, saw palmetto extract oil, olive oil, mineral oil, median chain tryglycerin, lavender oil, phosphatidylipids, rice bran oil, macadamia nut oil, arachis oil, phytosterol, phytotanol, or combinations thereof.

The filling composition may optionally comprise an oil from any edible oils such as, for example, fish oil, seal oil, borage oil, flax seed oil, evening primrose oil, soybean oil, safflower oil, sunflower oil, grape seed oil, squalene oil, squalane, sesame oil, hemp seed oil, pumpkin seed oil, or combinations thereof.

The present soft gel capsules may comprise other optional ingredients such as herbal extracts, vitamins, minerals, or other ingredients that benefit on human health, viscosity modifiers, process aids, lubricants, surfactants, and combinations thereof.

The present disclosure provides a process for making soft gel capsules comprising a probiotic. The process generally comprises the steps of selecting a suitable amount of gelatin or other gelling agent, selecting a suitable amount of plasticizer (if needed), selecting a suitable amount of starch (if needed), selecting a suitable amount of anti-tacking and softening agent (if needed), placing predetermined amounts of said selections into a cooking tank and adding a predetermined amount of water, mixing selections for at least 2 hours at an elevated temperature (e.g. 80-90° C.), reducing the temperature of the mixture in the cooking tank (e.g. to 55-65° C.) until all air bubbles are removed from the mixture, transferring the mixture from the cooking tank to a machine configured for producing soft gel capsules from the mixture, and then producing soft gel capsules from the mixture with the machine.

Beeswax is melted then mixed with oil. Silica and/or silica dioxide may be used as an alternative (or addition) to the beeswax. Silica or silica dioxide do not require heating prior to mixing with the oil. The other ingredients are then added including probiotic(s) and desiccant. Prefer the temperature of oil/beeswax mixture is at or lower than room temperature before adding probiotics. Preferably the probiotics are added after milling the paste if grinding need to apply in case of large particles in the ingredients. The process can also involve mixing the desiccant e.g. starch with heated or unheated oil, then adding melted beeswax to the mixture. The other ingredients are then added. Preferably the temperature of oil/beeswax mixture is at or lower than room temperature before adding probiotics. It is preferred to add the probiotics after milling the paste in case there are large particles in the ingredients. Optionally, it is preferred to use low water activity materials e.g. the oil in the composition.

It has been found that the present process proceeds with greater efficiency if the probiotic composition has a relatively small particles size. Therefore, it is preferred that the present probiotic composition pass through a US Mesh #80.

It is contemplated that any embodiment discussed in this specification can be implemented or combined with respect to any other embodiment, method, composition or aspect of the invention, and vice versa.

Unless defined otherwise, all technical and scientific terms used herein have the same meaning as is commonly understood by one of ordinary skill in the art to which this invention belongs. Unless otherwise specified, all patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. Citation of references herein is not to be construed nor considered as an admission that such references are prior art to the present invention.

Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

The present invention will be further illustrated in the following examples. However it is to be understood that these examples are for illustrative purposes only, and should not be used to limit the scope of the present invention in any manner.

### EXAMPLES

### Example 1

### Bifidobacterium

| | |
|---|---|
| (Bifilon-50T from Morinaga, Japan) | 8 billion cfu |
| Beeswax | 60 mg |
| Lecithin | 15 mg |
| Fish oil | 750 mg |
| Fish oil water activity was 0.15 | |

Fish oil was mixed with melted beeswax, lecithin and Bifidobacterium was added and mixed well. The mixture was ground by milling and passed through US mesh #80. Air bubbles were removed from the paste by vacuum and encapsulation. The soft gels were dried in the drying room at conditions of 20° C. and 19% RH for 8 hours to reach shell of soft gel moisture 6-8%. Probiotics was tested after three weeks storage at room temperature. 6 billion cfu per soft gel was observed.

### Example 2

### Bifidobacterium

| | |
|---|---|
| (Bifilon-50T from Morinaga, Japan) | 6 billion cfu |
| Beeswax | 50 mg |
| Lecithin | 10 mg |
| Seal oil | 750 mg |
| Tapioca starch | 105 mg |
| Seal oil water activity was 0.635 | |

The starch was preheated to dry, the seal oil was mixed with melted beeswax, and lecithin, starch and probiotics added to the paste at room temperature. The paste was ground by mill and passed through US mesh #80. Air bubbles were removed from the paste by vacuum and encapsulation. The soft gels were dried in the drying room under conditions of 27° C. and RH 40% for two and half days to reach shell moisture of soft gel 6-8%. Probiotics was tested after three weeks storage at room temperature. 2.4 billion cfu per soft gel was observed.

### Example 3

### Chewable Soft Gel Formula:

| BLIS K12 (Streptococcus salivarius) | 1.2 billion cfu | |
|---|---|---|
| Soybean oil | 146 | mg |
| Creamy orange flavour | 11.5 | mg |
| Beeswax | 30 | mg |
| Lecithin | 15 | mg |
| Sucralose | 0.36 | mg |
| Tapioca starch | 35 | mg |

Soybean oil water activity 0.3

The shell of chewable soft gel was prepared as described in U.S. Pat. No. 7,662,406. Starch was dried by heating overnight. Soybean oil and beeswax were heated to 65-80° C. and starch added with mixing, Lecithin and sucralose were added and mixed well. The formulation was ground by mill, and passed through US mesh #80, air bubbles were removed by vacuum, the creamy orange flavour and probiotics were added after paste temperature was below 25° C. The paste was encapsulated into soft gels. The soft gels were dried in a drying room with conditions of 20° C. and RH 20% for 2 hr to reach shell moisture content of 11%. The probiotics were tested after three weeks storage at room temperature. 0.59 billion cfu per soft gel was observed.

1. A soft gel capsule comprising:
a. an outer shell comprising gelatin; and
b. a filling comprising a probiotic and a desiccant.

2. A soft gel capsule according to claim 1, wherein the probiotic is selected from any probiotics that are benefit for human health.

3. A soft gel capsule according to claim 1, wherein the outer shell comprises about 20% to about 60% by weight of the outer shell composition of gelatin.

4. A soft gel capsule according to claim 1, wherein the outer shell comprises at least one plasticizer.

5. A soft gel capsule according to claim 1, wherein the outer shell comprises about 0.2% to about 15% by weight of the outer shell composition of plasticizer.

6. A soft gel capsule according to claim 1, wherein the outer shell comprises at least one anti-tack agent, water, at least one starch, or a combination thereof.

7. A soft gel capsule according to claim 1, wherein the probiotic is selected from Bifidobacterium adolescentis; Bifidobacterium animalis; Bifidobacterium bifidum; Bifidobacterium breve; Bifidobacterium infantis; Bifidobacterium lactis; Bifidobacterium longum; Lactobacillus acidophilus; Lactobacillus amylovorus; Lactobacillus casei; Lactobacillus fermentum; Lactobacillus gasseri; Lactobacillus johnsonii; Lactobacillus paracasei; Lactobacillus plantarum; Lactobacillus reuteri; Lactobacillus rhamnosus; Lactobacillus salivarius; Streptococcus thermophilus; Streptococcus Salivarius; Streptococcus oralis; Streptococcus uberis; Streptococcus rattus; Escherichia coli; Bacillus coagalans; Bacillus lausii; Saccharomyces cerevisiae; Saccharomyces boulardii; and combinations thereof.

8. A soft gel capsule according to claim 1, wherein the probiotic is selected from Bifidobacterium longum; Streptococcus salivarius; and combinations thereof.

9. A soft gel capsule according to claim 1, wherein the desiccant is selected from calcium sulfate; sodium sulfate; magnesium sulfate; dextrins; polysaccharides; oligosaccharides; lecithin; silicas; bentonite clays; starches; and combinations thereof.

10. A soft gel capsule according to claim 1, wherein the desiccant is selected from native starches.

11. A soft gel capsule according to claim 1, where the desiccant is selected from physically modified starches, chemically modified starches, and combinations thereof.

12. A soft gel capsule according to claim 1, where the desiccant is selected from potato starches, corn starches, wheat starches, oat starch, barley starch, rice starches, sorghum starches, legume starch, pea bean starch, tapioca starches, and combinations thereof.

13. A soft gel capsule according to claim 1, wherein the desiccant is selected from potato starches, corn starches, tapioca starches, and combinations thereof.

14. A soft gel capsule according to claim 1, wherein the filling comprises an emulsifier.

15. A soft gel capsule according to claim 1, wherein the filling comprises an emulsifier selected from lecithin, polysorbates, or combinations thereof.

16. A soft gel capsule according to claim 1, wherein the filling comprises one or more edible oils.

17. A soft gel capsule according to claim 1, wherein the filling comprises an oil selected from fish oil, krill oil, seal oil, borage oil, flax seed oil, evening primrose oil, soybean oil, hemp seed oil, pumpkin seed oil, grape seed oil, germ oil, safflower oil, squalene oil, squalane, sesame seed oil, arachidonic acid, conjugated linoleic acid, rosemary oil, lemon oil, peppermint oil, terrapin oil, pomegranate seed oil, sea buckthorn fruit oil, sunflower oil, chia oil, goji berry oil, sea buckthorn oil, jasmine oil, sweet almond oil, ginger oil, parsley seed oil, orange oil, halibut liver oil, wheat germ oil, aloe vera oil, garlic oil, avocado oil, castor oil, saw palmetto extract oil, olive oil, mineral oil, median chain tryglycerin, lavender oil, phosphatidylipids, rice bran oil, macadamia nut oil, arachis oil, phytosterol, phytotanol, or combinations thereof.

18. A soft gel capsule according to claim 1, wherein the filling comprises vitamins, minerals, plant extracts, or combination thereof.

19. A soft gel capsule according to claim 1, wherein the probiotic maintains at least about 50% of its colony forming units when compared to the probiotic before processing.

20. A process for producing a soft gel capsule, said process comprising:
a. Providing an outer shell composition comprising a gelling agent;
b. Providing a filling composition comprising a probiotic and a desiccant;
c. Forming a soft gel capsule from the outer shell composition; and
d. Introducing the filling composition to the inside of the outer shell composition.
